(19) United States
(12) Patent Application Publication
(10) Pub. No.: US 2004/0092844 A1
(43) Pub. Date: May 13, 2004

(54) Guide catheter

(76) Inventors: Eric T. Johnson, Temecula, CA (US); Cindy Sherman, Temecula, CA (US)

Correspondence Address: BLAKELY SOKOLOFF TAYLOR & ZAFMAN, 12400 WILSHIRE BOULEVARD, SEVENTH FLOOR, LOS ANGELES, CA, 90025, US

(21) Appl. No.: 10/293,535

(22) Filed: Nov. 12, 2002

(51) Int. Cl. A61M 25/00

(52) U.S. Cl. 600/585; 604/524

(57) ABSTRACT

An apparatus that includes a tubular body portion having dimensions suitable for insertion into a human blood vessel as a guide catheter, the tubular body portion having a proximal end and a distal end and a lumen therethrough, where the distal portion of the tubular body portion includes a first convex curved portion, a concave curved portion distal to the first convex curved portion, and a second convex curved portion distal to the concave curved portion.

### BACKGROUND

1. Field of the Invention

This invention relates to cardiovascular guide catheters.

2. Description of Related Art

Generally, cardiovascular guide catheters are known in the art. Guide catheters may be used to provide a guide through a vasculature of a patient into which another catheter or device may be inserted. U.S. Pat. No. 4,927,412 discloses a coronary sinus catheter having a flexible elongate member with proximal and distal extremities.

U.S. Pat. No. 5,021,045 discloses a retrograde cardioplegia catheter.

U.S. Pat. No. 5,226,427 discloses a stylet for use with a retrograde cardioplegia catheter.

U.S. Pat. No. 5,385,548 discloses a catheter for retrograde perfusion of the heart through the coronary sinus.

U.S. Pat. No. 5,597,377 discloses a catheter for retroperfusion of myocardium.

U.S. Pat. No. 5,620,418 discloses a coronary sinus catheter.

U.S. Pat. No. 5,662,607 discloses a catheter for supplying liquid to the coronary sinus.

U.S. Pat. No. 5,707,358 discloses a perfusion catheter for use in open heart surgery.

U.S. Pat. No. 5,720,726 discloses a catheter for retrograde perfusion of the heart through the coronary sinus.

U.S. Pat. No. 5,779,685 discloses a retrograde cardioplegia catheter for the delivery of a cardioplegia solution to a patient's heart.

U.S. Pat. No. 5,807,326 discloses a coronary sinus catheter assembly for the retrograde infusion of cardioplegia solutions into the coronary sinus.

U.S. Pat. No. 5,879,499 discloses a process for forming a multilumen catheter.

U.S. Pat. No. 5,913,842 discloses a retrograde delivery catheter.

U.S. Pat. No. 5,916,193 discloses a venting catheter, system and method for withdrawing blood and other fluids from a patient's heart.

U.S. Pat. No. 5,967,988 discloses a retrograde coronary sinus perfusion catheter that includes a flexible, tubular catheter body and an inflatable balloon.

U.S. Pat. No. 6,071,271 discloses a catheter system for use in minimally invasive cardiac surgical procedures.

U.S. Pat. No. 6,241,699 discloses a catheter system and method of performing posterior epicardial revascularization and intracardiac surgery on a beating heart.

U.S. Pat. No. 6,340,356 discloses an intraluminal catheter having an expandable tubular open-walled element for immobilizing at least part of the catheter within a patient's body lumen.

U.S. patent application Ser. No. 09/822,678, which published as Publication No. 2001/0,044,624, discloses a combination method and system for intravascularly accessing and visualizing a body structure.

### SUMMARY

In one embodiment, there is disclosed a guide catheter that includes a first convex curved portion, a concave curved portion distal to the convex curved portion, and a second convex curved portion distal to the concave curved portion.

In another embodiment, there is disclosed a method of producing a guide catheter that includes providing a mandrel having at least one concave curved portion and at least one convex curved portion, covering the mandrel with a liner, wrapping a plurality of ribbons and/or wires exterior to the liner, and placing a matrix exterior to the plurality of ribbons and/or wires.

In another embodiment, there is disclosed a catheter kit that includes a guide catheter that includes a first convex curved portion, a concave curved portion distal to the first convex curved portion, and a second convex curved portion distal to the concave curved portion, a proximal end, and a distal end, wherein the guide catheter is configured so that the distal end of the guide catheter can be placed in an opening of a coronary sinus when fed through an inferior vena cava; a guidewire adapted to be received within the guide catheter; and a delivery catheter adapted to be received within the guide catheter.

In general, the curved portions of the guide catheter enable ease of placement of the guide catheter according to one contemplated use in the opening of the coronary sinus when fed through the inferior vena cava.

Additional features, embodiments, and benefits will be evident in view of the figures and detailed description presented herein.

### BRIEF DESCRIPTION OF THE DRAWINGS

The features, aspects, and advantages of the invention will become more thoroughly apparent from the following detailed description, appended claims, and accompanying drawings in which:

FIG. 1 schematically illustrates major arteries of the body;

FIG. 2 schematically illustrates major veins of the body;

FIG. 3 schematically illustrates a view of the heart;

FIG. 4 schematically illustrates a view of the heart with a guide catheter inserted;

FIG. 5 schematically illustrates a cross-sectional view of the guide catheter of FIG. 4;

FIG. 6 schematically illustrates a view of the heart with a guide catheter inserted therein;

FIG. 7 schematically illustrates a view of the heart with a guide catheter inserted therein;

FIG. 8 schematically illustrates a guide catheter;

FIG. 9 schematically illustrates a cross-sectional view of the guide catheter of FIG. 8;

FIG. 10 schematically illustrates a cross-sectional view of a guide catheter; and

FIG. 11 schematically illustrates a method of manufacturing a guide catheter.

### DETAILED DESCRIPTION

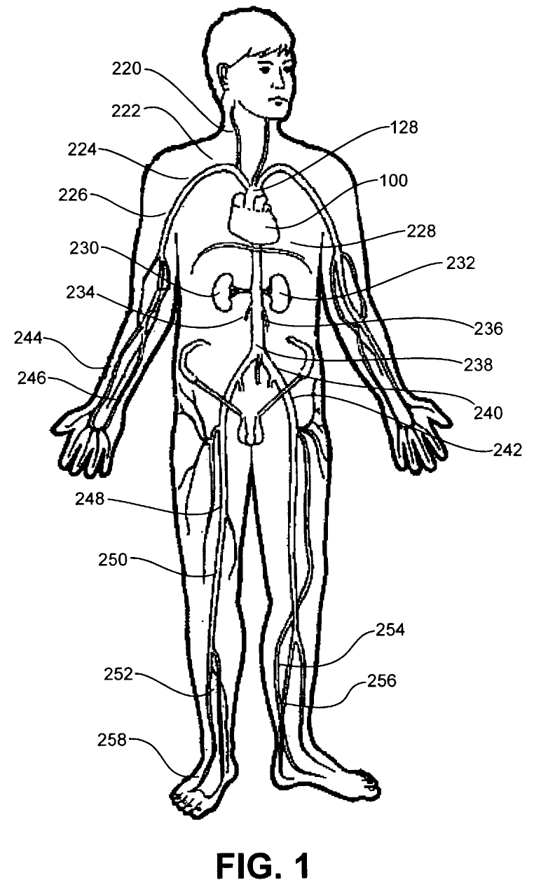

Referring now to FIG. 1, which schematically illustrates major arteries of the body. The oxygenated blood of the body originates in heart 100 and is pumped by the left ventricle (not shown) into aorta 128. From aorta 128, the blood branches throughout the rest of the body. A first branch, subclavian artery 222 feeds axillary artery 224 which turns into brachial artery 226. Brachial artery 226 in turn feeds radial artery 244 and ulnar artery 246. Another branch off of aorta 128 is common carotid artery 220. Diaphragm 228 is also shown under heart 100. The lower branch of aorta 238 feeds blood to kidneys 230 through renal artery 232. There are also two branches off of the lower branch of aorta 238, the first, superior mesenteric artery 234, and the second, inferior mesenteric artery 236. Lower branch of aorta 238 splits and feeds common iliac artery 240 for both legs. The common iliac artery in turn branches into external iliac artery 242 and femoral artery 248. One of the branches of femoral artery 248 is popliteal artery 250 which branches into anterior tibial artery 252, posterior tibial artery 256, and dorsalis pedis artery 258. Peroneal artery 254 branches off of external iliac artery 242.

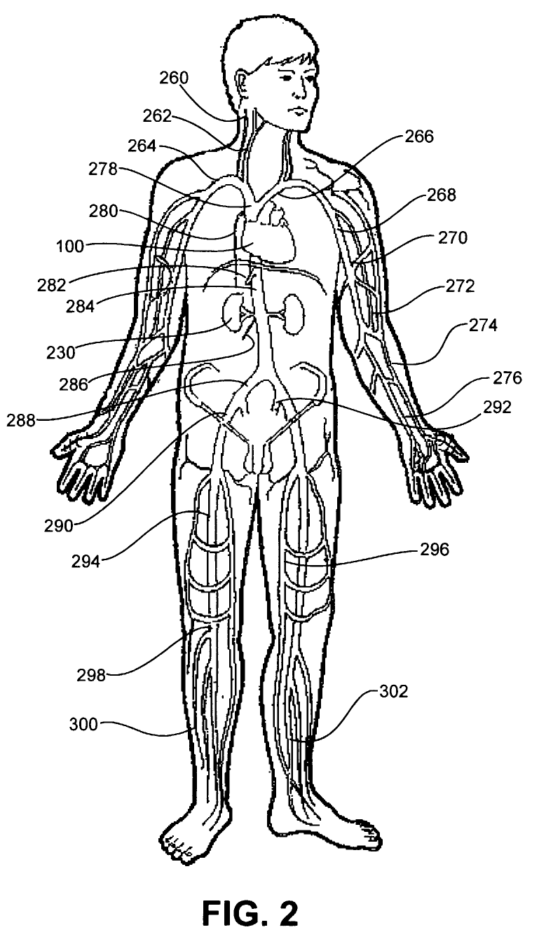

Referring now to FIG. 2, which schematically illustrates major veins of the body. The deoxygenated blood returns to heart 100 through the venous system. Some blood returning from the legs flows into posterior tibial vein 302, and anterior tibial vein 300, which feed into popliteal vein 298, and flows into femoral vein 294. Another vein in the legs is great saphenous vein 296 which also feeds into femoral vein 294. Blood then flows into either internal iliac vein 292 or external iliac vein 290 which then flow into common iliac vein 288 to return to heart 100 via inferior vena cava 284. Blood returning from kidneys 230 flows through renal vein 286 and then to inferior vena cava 284 which mixes with the blood coming from common iliac vein 288. Other branches feeding into inferior vena cava 284 include hepatic vein 282. Blood returning from the left arm flows into ulnar vein 276, radial vein 274, or brachial vein 272, or basilic vein 270, and flows into axillary vein 268. Blood flows from axillary vein 268 into left innominate vein 266 which flows into superior vena cava 278. (A similar arrangement can be found in the right arm.) Blood also flows into superior vena cava 278 from right subclavian vein 264, and from external jugular vein 260 and internal jugular vein 262.

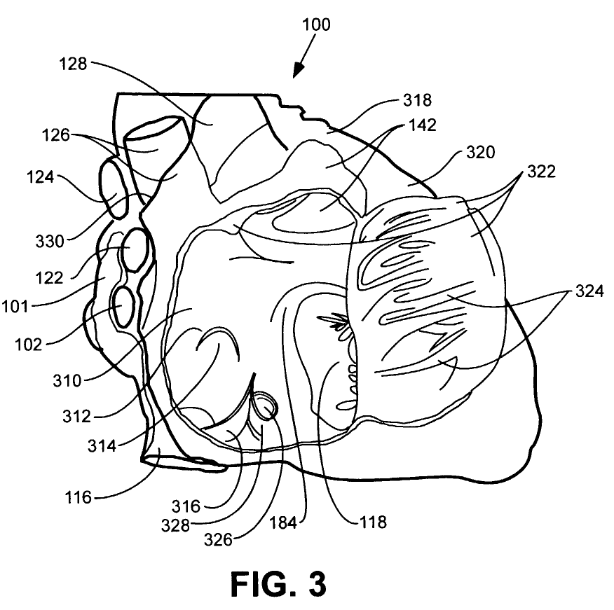

Referring now to FIG. 3 is a right lateral view of heart 100 with an opened right atrium. Heart 100 includes aorta 128, superior vena cava 126, right pulmonary artery 124, pericardial reflection 330, pulmonary veins 102, left atrium 101, right atrium 122, inferior vena cava 116, conus arteriosis 320, right auricle 142, and pulmonary trunk 318. The exterior wall of right atrium 122 has been dissected to reveal interatrial septum 310, limbus of fossa ovalis 312, fossa ovalis 314, eustachian valve of inferior vena cava 316, opening of coronary sinus 326, thebesian valve of coronary sinus 328, pectinate muscles 324, tricuspid valve 118, atrioventricular part of membranous septum 184, and crista terminalis 322.

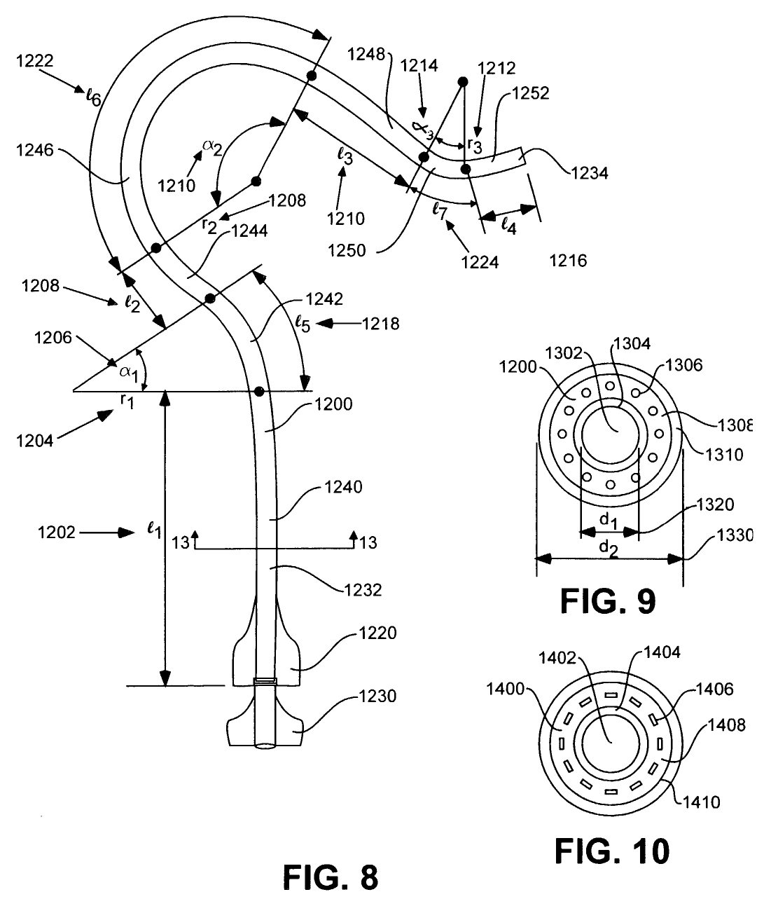

Referring now to FIG. 8, guide catheter 1200 is shown. Guide catheter 1200 includes proximal end 1232 and distal end 1234. Adjacent proximal end 1232 is optionally provided turning device 1230 and transition 1220 between guide catheter 1200 and optional turning device 1230.

In one embodiment, starting at proximal end 1232 of guide catheter 1200 is straight portion 1240 having length l₁ labeled with reference numeral 1202. A representative length for length l₁ to be advanced from femoral vein (294, see FIG. 2) to coronary sinus is between about 30 and about 90 cm, and in another embodiment between about 45 and 75 cm. Distal to straight portion 1240 is first optional convex curved portion 1242 having arc length l₅ labeled with reference numeral 1218. Optional convex curved portion 1242 has radius of curvature r₁ 1204 and arc angle α₁ 1206. Distal to optional convex curved portion 1242 is straight portion 1244 having length l₂ 1208. Distal to straight portion 1244 is concave curved portion 1246 having arc length l₆ 1222. Concave curved portion 1246 has radius of curvature r₂ 1208 and arc angle α₂ 1210. Distal to concave curved portion 1246 is straight portion 1248 having length l₃ 1210. Distal to straight portion 1248 is optional convex curved portion 1250 having arc length l₇ 1224. Optional convex curved portion 1250 has radius of curvature r₃ 1212 and arc angle α₃ 1214. Distal to optional convex curved portion 1250 is optional straight portion 1252 having length l₄ 1216.

In one embodiment, sum of lengths l₁ 1202, l₂ 1208, l₃ 1210, l₄ 1216, l₅ 1218, l₆ 1222, and l₇ 1224 is between about 30 and about 120 cm, and in another embodiment between about 45 and 90 cm.

In one embodiment, length l₁ 1202 is between about 15 and about 105 cm. In another embodiment, arc length l₅ 1218 is between about 5 and about 25 cm. In another embodiment, radius of curvature r₁ 1204 is between about 10 and about 50 cm. In another embodiment, arc angle α₁ 1206 is between about 5° and about 45°. In anther embodiment, length l₂ 1208 is between about 1 and about 10 cm. In another embodiment, arc length l₆ 122 is between about 5 and about 30 cm. In another embodiment, radius of curvature r₂ 1208 is between about 3 and about 20 cm. In another embodiment, arc angle α₂ 1210 is between about 60° and about 210°. In another embodiment, length l₃ 1210 is between about 1 and about 10 cm. In another embodiment, arc length l₇ 1224 is between about 2 and about 10 cm. In another embodiment, radius of curvature r₃ 1212 is between about 5 and about 20 cm. In another embodiment, arc angle α₃ 1214 is between about 15° and about 45°. In another embodiment, length l₄ 1216 is between about 1 and about 10 cm.

Referring now to FIG. 9, is a cross-sectional view of catheter 1200 of FIG. 8 taken along line 13-13. Catheter 1200 has lumen 1302 defining its interior. Exterior to lumen 1302 is inner liner 1304. Exterior to inner liner 1304 is a composite of a plurality of wires 1306 in matrix 1308. Exterior to wires 1306 and matrix 1308 is optional outer covering 1310.

In one embodiment, inner liner 1304 includes a polymer. Inner liner 1304 may include Teflon® (registered trademark of DuPont de Nemours and Company of Wilmington, Del.), polytetrafluoroethylene (PTFE), and/or fluorinated ethylene-propylene resins (FEP).

In one embodiment, wires 1306 may include a metal or a polymer. In one embodiment, wires 1306 include stainless steel, for example stainless steel 304, and/or a stainless steel having a tensile strength of about 150 to about 350 thousand pounds per square inch (ksi). In another embodiment, wires 1306 may include tungsten and/or bismuth bicarbonate.

In one embodiment, matrix 1308 may include a polymer. In another embodiment, matrix 1308 may include a thermoset polymer. In another embodiment, matrix 1308 may include polyamide, PEBAX, nylon, urethane, and/or polyurethane.

Catheter 1200 has inner diameter d₁ 1320, and outer diameter d₂ 1330. In one embodiment, inner diameter d₁ 1320 is between about 1 and about 5 millimeters. In another embodiment, inner diameter d₁ 1320 is between about 2 and about 4 millimeters. In another embodiment, inner diameter d₁ 1320 is about 3 millimeters. In one embodiment, outer diameter d₂ 1330 is between about 1 and about 5 millimeters. In another embodiment, outer diameter d₂ 1330 is between about 2 and about 4 millimeters. In another embodiment, outer diameter d₂ 1330 is about 3 millimeters.

Another embodiment is illustrated in FIG. 10. Catheter 1400 has lumen 1402 defining its interior. Exterior to lumen 1402 is inner liner 1404. Exterior to inner liner 1404 is a composite of plurality of ribbons 1406 in matrix 1408. Exterior to ribbons 1406 and matrix 1408 is optional outer covering 1410.

In one embodiment, inner liner 1404 includes a polymer. Inner liner 1404 may include Teflon® (registered trademark of DuPont), polytetrafluoroethylene (PTFE), and/or fluorinated ethylene-propylene resins (FEP).

In one embodiment, ribbons 1406 may include a metal or a polymer. In one embodiment, ribbons 1406 include stainless steel, for example stainless steel 304, and/or a stainless steel having a tensile strength of about 150 to about 350 ksi. In another embodiment, ribbons 1406 may include tungsten and/or bismuth bicarbonate.

In one embodiment, matrix 1408 may include a polymer. In another embodiment, matrix 1408 may include a thermoset polymer. In another embodiment, matrix 1408 may include polyamide, polyether-block co-polyamide (PEBAX), nylon, urethane, and/or polyurethane.

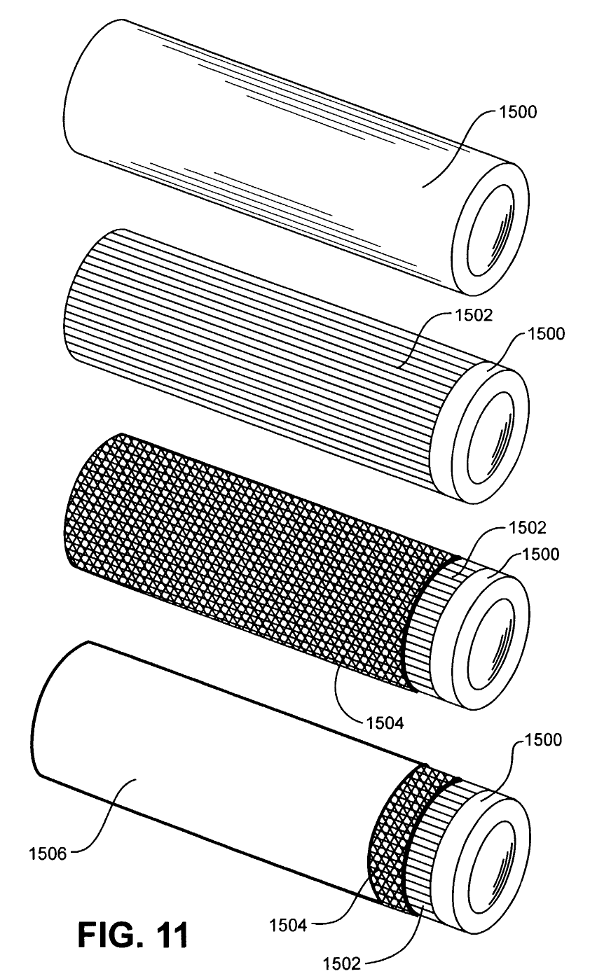

One embodiment of manufacturing a guide catheter is illustrated in FIG. 11. Mandrel 1500 is covered with liner 1502. Plurality of ribbons and/or wires 1504 is placed exterior to liner 1502. In one embodiment, ribbons and/or wires 1504 are wound in a helical fashion. In another embodiment, ribbons and/or wires 1504 are weaved in a helical fashion so that some ribbons or wires are in one direction and some ribbons or wires are in another direction, for example clockwise and counter-clockwise. In one embodiment, ribbons and/or wires 1504 are weaved in a helical fashion having a wind angle measured from an axis of mandrel 1500 between about 30 and about 60 degrees. In another embodiment, ribbons and/or wires 1504 are weaved in a helical fashion having a wind angle measured from an axis of mandrel 1500 of about 45 degrees. In another embodiment, ribbons and/or wires 1504 are substantially parallel to an axis of mandrel 1500. Matrix 1506 is placed over plurality of ribbons and/or wires 1504. In one embodiment, matrix 1506 is a thermoset or a partial thermoset polymer.

In one embodiment, mandrel 1500 has a shape that includes a plurality of curves and a plurality of straight portions, where at least one curve is concave and at least one curve is convex. In one embodiment, the guide catheter may be baked following the application of matrix 1506 over plurality of ribbons and/or wires 1504.

In one embodiment, mandrel 1550 has a first convex curved portion, a concave curved portion distal to the first convex curved portion, and a second curved distal to the concave curved portion; following the application of matrix 1506, plurality of ribbons and/or wires 1504, mandrel 1500, liner 1502, plurality of ribbons and/or wires 1504, and matrix 1506 may be baked so that the guide catheter produced by this process has a shape having a first convex curved portion, a concave curved portion distal to first convex curved portion, and a second curved portion distal to concave curved portion.

In one embodiment, matrix 1506 includes one or more of a polymer, a thermoset polymer, a polyamide, a nylon, and/or a urethane, and/or mixtures thereof.

In one embodiment, matrix 1506 is a material with a shape memory that will retain the curved portions after being removed from the mandrel. In another embodiment, plurality of ribbons and/or wires 1504 includes a material with a shape memory that will retain the curved portions after being removed from the mandrel.

In one embodiment, plurality of ribbons and/or wires 1504 includes a shape memory alloy, an intermetallic, a nitinol, and/or a nickel-titanium alloy.

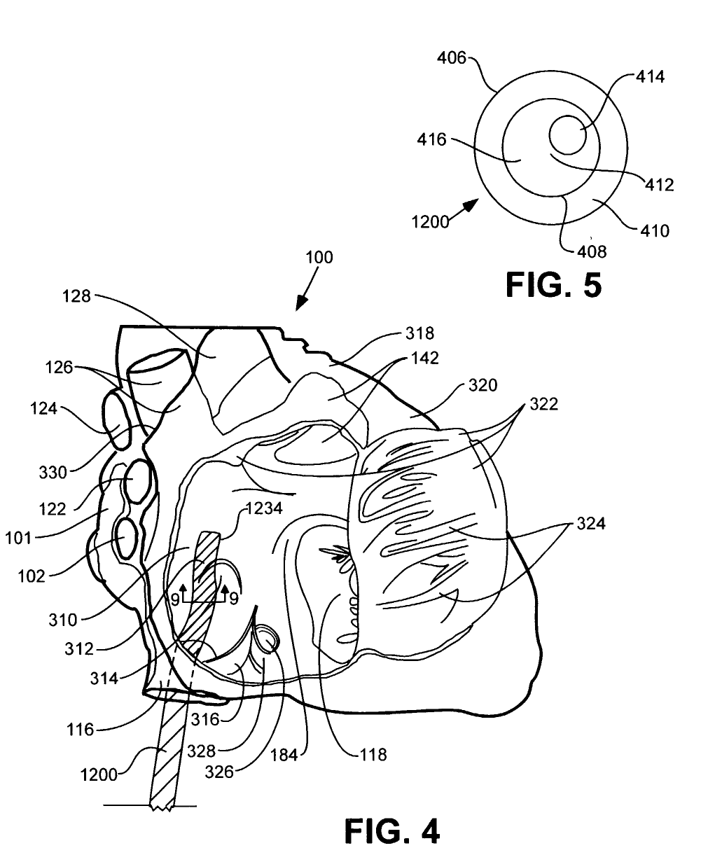

Referring now to FIG. 4, which schematically illustrates a similar view of heart 100 as FIG. 3. Guide catheter 1200 having a guidewire (not shown), and having distal end 1234 and proximal end (not shown) has been advanced through inferior vena cava 116 and into right atrium 122. One way this may be accomplished is by introducing guide catheter 1200 having a guidewire (not shown) through femoral vein (294, see FIG. 2), advancing guide catheter 1200 through common iliac vein 288, through inferior vena cava 116 (284 in FIG. 2) and into right atrium 122.

Referring now to FIG. 5, which schematically illustrates a cross-sectional view of guide catheter 1200 of FIG. 4 taken along the line 9-9. Guide catheter 1200 has outer wall 406, inner wall 408, and catheter body 410 between outer wall 406 and inner wall 408. Lumen 412 is interior to inner wall 408. Guidewire 414 is received within lumen 412 of guide catheter 1200. In one embodiment, guidewire 414 does not occupy entire lumen 412 and leaves annular space 416 about guidewire 414. In another embodiment, guidewire 414 is sized to occupy substantially entire lumen 412, so that annular space 416 has a negligible size.

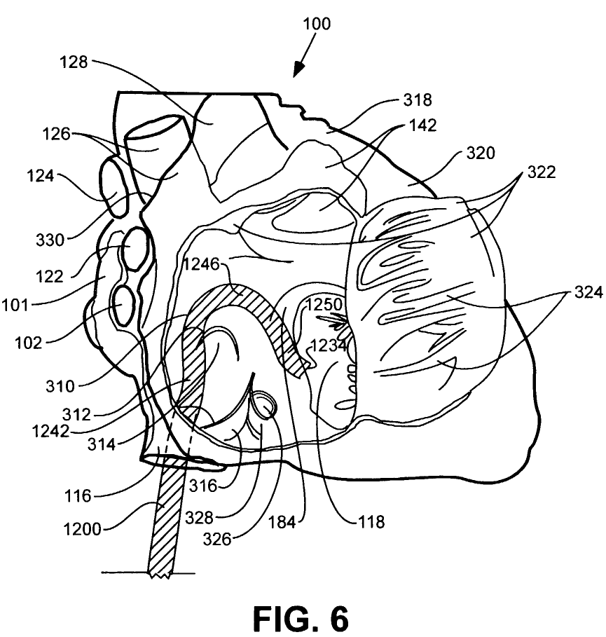

Referring now to FIG. 6, is heart 100 with guide catheter 1200 fed into right atrium 122 through inferior vena cava 116. Guide catheter 1200 no longer has guidewire 414 therein so that guide catheter 1200 adopts a shape with first curve 1242, second curve 1246, and optional third curve 1250. From distal end 1234, first curve 1242 is located proximal to second curve 1246 and third curve 1250. Second curve 1246 is proximal to third curve 1250. First curve 1242 and third curve 1250 are opposite one another. In one embodiment, guide catheter (not shown) has only first curve 1242 and second curve 1246. In one embodiment, first curve 1242, second curve 1246, and optional third curve 1250 are each selected to facilitate placement of distal end 1234 of guide catheter 1200 into opening of coronary sinus 326 so that distal end 1234 may be located within the coronary sinus (not shown).

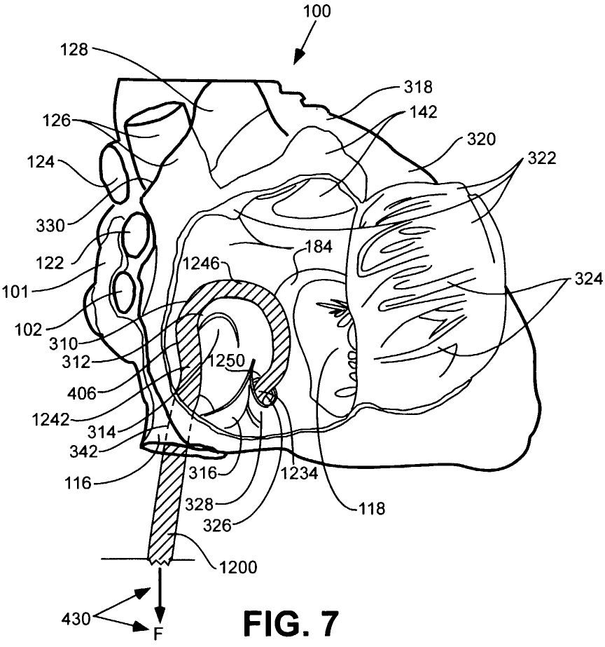

Referring now to FIG. 7, heart 100 is shown. Guide catheter 1200 is shown advanced through inferior vena cava 116 into right atrium 122 for placement into opening of coronary sinus 326. Guide catheter 1200 has distal end 1234 adjacent to opening of coronary sinus 326. In one embodiment, third curve 1250 adjacent to distal end 1234 of guide catheter 1200 serves to position distal end 1234 so that distal end 1234 opens thebesian valve 328 of the coronary sinus. In one embodiment, second curve 1246 of guide catheter 1200 serves to translate force F 430 marked by an arrow to position distal end 1234 of guide catheter 1200 into the coronary sinus (not shown) through opening of coronary sinus 326. In one embodiment, first curve 1242 of guide catheter 1200 serves to orient guide catheter 1200 so that outside wall 406 of guide catheter 1200 can anchor against free wall 340 of right atrium 122 and/or inside wall 342 of inferior vena cava 116 in order to position distal end 1234 of guide catheter 1200 within opening of coronary sinus 326. When force F 430 is applied to guide catheter 1200, the shape of guide catheter 1200 with first curve 1242, second curve 1246, and third curve 1250, forces distal end 1234 into opening of coronary sinus 326. If guide catheter 1200 only has second curve 1246, as force F 430 was applied, distal end 1234 would tend to be pulled away from coronary sinus opening 326, since the force F 430 could not translate to distal end 1234. However, since guide catheter 1200 is provided with first curve 1242, second curve 1246, and third curve 1250, force F 430 is transmitted to distal end 1234 by reaction forces provided by free wall 340 of right atrium 122 and/or inside wall 342 of inferior vena cava 116, and through first curve 1242, second curve 1246, and third curve 1250 to position distal end 1234 within opening of coronary sinus 326.

Referring again to FIG. 2, a guide catheter may be inserted into posterior tibial vein 302, interior tibial vein 300, popliteal vein 298, or femoral vein 294. In another embodiment, a guide catheter may be fed into great saphenous vein 296, which feeds into femoral vein 294. A catheter inserted into one of the veins of the leg, feeds into femoral vein 294, which then feeds into external iliac vein 290, which then feeds into common iliac vein 288 to access heart 100 via inferior vena cava 284. In another embodiment, a guide catheter may be fed directly into external iliac vein 290 or common iliac vein 288. In another embodiment, a guide catheter may be feed into hepatic vein 282 or directly into inferior vena cava 284 to access heart 100.

In one method of use, a medical professional (not shown) can make an incision (not shown) in femoral vein 294 (shown in FIG. 2), and insert guide catheter 1200. Distal end 1234 may be fed into femoral vein 294. Proximal end (for example, 1232) of guide catheter 1200 may be provided with a mechanism for steering (for example turning device 1230 which may be attached or coupled to proximal end of guide catheter 1200). After being inserted into femoral vein 294, guide catheter 1200 is fed into external iliac vein 290, then fed into common iliac vein 288, then fed into inferior vena cava 284 (shown in FIG. 2). Once in inferior vena cava (284 in FIG. 2, 166 in FIG. 3), the medical professional can feed guide catheter 1200 into heart 100. Specifically, the medical professional can feed guide catheter 1200 into inferior vena cava 166, then into right atrium 122 (shown in FIG. 3). Once distal end 1234 is in right atrium 122, the medical professional can remove the guidewire (not shown) so that guide catheter 1200 maintains its natural shape with at least two curves, or, at least three curves. The entire process of feeding guide catheter 1200 into right atrium 122 may be accomplished under x-ray or fluoroscopy, for example. After guide catheter 1200 has maintained its regular shape, for example, having three curves, the medical professional positions guide catheter 1200 using a turning device (for example, 1230) and advancing guide catheter 1200 proximally or distally to align distal end 1234 adjacent to thebesian valve 328 near opening of coronary sinus 326 (shown in FIG. 3). Once distal end 1234 is in position, guide catheter 1200 can be rotated using a turning device (for example, 1230) to force distal end 1234 into opening of coronary sinus 326 past thebesian valve 328 (shown in FIG. 3). Once distal end 1234 is inside the opening of coronary sinus 326, the medical professional can pull guide catheter or can advance guide catheter 1200 proximally, or exert a proximal force (for example, pull on guide catheter) to force distal end 1234 further into opening of coronary sinus 326. Once guide catheter 1200 has been positioned within opening of coronary sinus 326, the medical professional can secure the location (for example, with sutures) of guide catheter 1200 at the incision in femoral vein 294, and then can advance other catheters or devices through guide catheter 1200 and into the coronary sinus.

In one embodiment, guide catheter 1200 construction (for example, steel braid, matrix transitions, liner) may be balanced to minimize “whip.” This construction is to avoid whip, which is a control issue where the tip of the catheter will not aim well or be controlled.

In one embodiment, there is disclosed an apparatus that includes a tubular body portion having dimensions suitable for insertion into a human blood vessel as a guide catheter, the tubular body portion having a proximal end and a distal end, and a lumen therethrough, the distal portion of the tubular body portion including a first convex curved portion, a concave portion distal to the first convex curved portion, and a second convex curved portion distal to the concave curved portion.

In another embodiment, there is disclosed an apparatus having a proximal end and a distal end, where a first convex curved portion, a concave curved portion, and a second convex curved portion define an arc segment, the arc segment having dimensions so that the distal end of the apparatus can be placed in an opening of a coronary sinus when fed through an inferior vena cava.

In another embodiment there is disclosed an apparatus that includes a plurality of wires in a thermoset polymer matrix.

In another embodiment, there is disclosed an apparatus that includes a plurality of ribbons in a thermoset polymer matrix.

In another embodiment, there is disclosed a plurality of wires that include stainless steel and/or that are made of stainless steel.

In another embodiment, there is disclosed an apparatus having a plurality of wires, where the wires include a material that is not visible under MRI, and/or where the plurality of wires are made of a material not visible under MRI, and/or where the plurality of wires are not visible under MRI. In another embodiment, there is disclosed an apparatus with a plurality of wires, where the plurality of wires are adapted to be used with MRI visioning systems.

In another embodiment, there is disclosed an apparatus including one or more wires, where the wires include or are made of a material having a tensile strength of about 150 ksi to about 350 ksi, and/or where the wires have a tensile strength of about 150 ksi to about 350 ksi.

In another embodiment, there is disclosed an apparatus having a plurality of wires, where there is between about 3 and about 10 wires in a clockwise helical rotation, and between about 3 and about 10 wires in a counterclockwise helical rotation about the apparatus.

In another embodiment, there is disclosed an apparatus having a number of ribbons, where the number of ribbons is between about 6 and about 24 ribbons.

In another embodiment, there is disclosed an apparatus having between about 8 and about 24 ribbons, with about 4 to about 12 of the ribbons in a clockwise helical rotation, and about 4 to about 12 of the ribbons in a counterclockwise helical rotation about the apparatus.

In another embodiment, there is disclosed an apparatus that includes a number of ribbons, where the ribbons include stainless steel, and/or the ribbons are made of stainless steel.

In another embodiment, there is disclosed an apparatus that includes a first guide catheter having a lumen therethrough, where the lumen includes a second guide catheter. In another embodiment, the second guide catheter is adapted to change the shape of the first guide catheter.

In another embodiment, there is disclosed an apparatus that includes a thermoset polymer matrix. In another embodiment, the thermoset polymer is selected from polyamides, polyether block amides, nylon-6, nylon-12, urethane, polyurethane, silicone, and/or blends and mixtures thereof.

In another embodiment, there is disclosed a method of producing a guide catheter that includes providing a mandrel having at least one concave curve portion and at least one convex curve portion, covering the mandrel with a liner, wrapping a plurality of ribbons and/or wires exterior to the liner, and placing a matrix exterior to the ribbons and/or wires.

In another embodiment, there is disclosed a method of making a guide catheter, where the guide catheter has a matrix that includes a thermoset polymer and/or a partial thermoset polymer.

In another embodiment, there is disclosed a method of producing a guide catheter that includes baking the guide catheter to set a shape having at least one concave curved portion and at least one convex curved portion.

In another embodiment there is disclosed a method of producing a guide catheter, that includes wrapping a plurality of ribbons and/or wires about a liner, where there is between about 3 and about 10 ribbons wrapped in a clockwise helical rotation exterior to a liner, and between about 3 and about 10 ribbons wrapped in a counterclockwise helical rotation exterior to a liner.

In another embodiment, there is disclosed a catheter kit that includes a guide catheter that has a first convex curved portion, a concave curved portion distal to the first convex curved portion, and a second convex curved portion distal to the concave curved portion, a proximal end, and a distal end, where the guide catheter is configured so that the distal end of the guide catheter can be placed in an opening of a coronary sinus when fed through an inferior vena cava; a guidewire adapted to be received within the guide catheter; and a delivery catheter adapted to be received within the guide catheter.

In the preceding detailed description, the invention is described with reference to specific embodiments thereof. It will, however, be evident that various modifications and changes may be made thereto without departing from the broader spirit and scope of the invention as set forth in the claims. The specification and drawings are, accordingly, to be regarded in an illustrative rather than a restrictive sense.

What is claimed is:

1. An apparatus comprising:
a tubular body portion having dimensions suitable for insertion into a human blood vessel as a guide catheter, the tubular body portion having a proximal end and a distal end and a lumen therethrough, the distal portion of the tubular body portion comprising:
a first convex curved portion;
a concave curved portion distal to the first convex curved portion; and
a second convex curved portion distal to the concave curved portion.

2. The apparatus of claim 1 further comprising:
a proximal end and a distal end;
wherein the first convex curved portion, the concave curved portion, and the second convex curved portion define an arc segment, the arc segment having dimensions so that the distal end of the apparatus can be placed in an opening of a coronary sinus when fed through an inferior vena cava.

3. The apparatus of claim 1, further comprising a plurality of wires in a thermoset polymer matrix.

4. The apparatus of claim 1, further comprising a plurality of ribbons in a thermoset polymer matrix.

5. The apparatus of claim 3, wherein the plurality of wires comprise stainless steel.

6. The apparatus of claim 3, wherein the plurality of wires comprise a material not visible under MRI.

7. The apparatus of claim 3, wherein the plurality of wires are adapted to be used with MRI Visioning Systems.

8. The apparatus of claim 3, wherein the wires comprise a material having a tensile strength of about 150 ksi to about 350 ksi.

9. The apparatus of claim 3, wherein the plurality of wires comprise between about 3 and about 10 wires in a clockwise helical rotation and between about 3 and about 10 wires in a counterclockwise helical rotation.

10. The apparatus of claim 4, wherein the plurality of ribbons comprise between about 6 and about 24 ribbons.

11. The apparatus of claim 4, wherein the plurality of ribbons comprise between about 8 and about 24 ribbons, about 4 to about 12 of the ribbons in a clockwise helical rotation, and about 4 to about 12 of the ribbons in a counterclockwise helical rotation.

12. The apparatus of claim 4, wherein the ribbons comprise stainless steel.

13. The apparatus of claim 1, wherein the apparatus comprises a first guide catheter further comprising a lumen therethrough, the lumen comprising a second guide catheter.

14. The apparatus of 13, wherein the second guide catheter is adapted to change the shape of the first guide catheter.

15. The apparatus of claim 3, wherein the thermoset polymer matrix comprises a polymer selected from the group consisting of polyamide, polyether block amide, nylon-6, nylon-12, polyurethane, silicone, and blends and mixtures thereof.

16. The apparatus of claim 4, wherein the thermoset polymer matrix comprises a polymer selected from the group consisting of polyamide, polyether block amide, nylon-6, nylon-12, polyurethane, silicone, and blends and mixtures thereof.

17. A method of producing a guide catheter comprising:
providing a mandrel having at least one concave curved portion and at least one convex curved portion;
covering the mandrel with a liner;
wrapping a plurality of at least one of ribbons and wires exterior to the liner; and
placing a matrix exterior to the plurality of at least one of ribbons and wires.

18. The method of claim 17 wherein the matrix comprises at least one of a thermoset polymer and a partial thermoset polymer.

19. The method of claim 17 wherein the plurality of at least one of ribbons and wires comprises a plurality of ribbons.

20. The method of claim 17 further comprising baking the guide catheter to set a shape having at least one concave curved portion and at least one convex curved portion.

21. The method of claim 17 wherein the wrapping the plurality of at least one of ribbons and wires comprises wrapping between about 3 and about 10 ribbons in a clockwise helical rotation exterior to liner, and wrapping between about 3 and about 10 ribbons in a counter-clockwise helical rotation exterior to liner.

22. The method of claim 17 wherein the matrix comprises a polymer selected from the group consisting of polyamide, polyether block amide, nylon-6, nylon-12, polyurethane, silicone, and blends and mixtures thereof.

23. A catheter kit comprising:
a guide catheter comprising a first convex curved portion, a concave curved portion distal to the first convex curved portion, and a second convex curved portion distal to the concave curved portion, a proximal end, and a distal end, wherein the guide catheter is configured so that the distal end of the guide catheter can be placed in an opening of a coronary sinus when fed through an inferior vena cava;
a guide wire adapted to be received within the guide catheter; and
a delivery catheter adapted to be received within the guide catheter.
